(19) United States
(12) Patent Application Publication
(10) Pub. No.: US 2026/0227379 A1
(43) Pub. Date: Aug. 6, 2026

(54) DEVICES AND METHODS FOR CLOT BURST PRESSURE MEASUREMENT

(71) Applicant: THE BOARD OF REGENTS OF THE UNIVERSITY OF TEXAS SYSTEM, Austin, TX (US)

(72) Inventors: Brijesh S. GILL, Houston, TX (US); Atharwa R. MANKAME, Houston, TX (US); Julia LITVINOV, Houston, TX (US); Charles S. COX, Houston, TX (US)

(73) Assignee: THE BOARD OF REGENTS OF THE UNIVERSITY OF TEXAS SYSTEM, Austin, TX (US)

(21) Appl. No.: 19/154,013

(22) PCT Filed: Feb. 5, 2024
(86) PCT No.: PCT/US2024/014387

§ 371 Date: Aug. 6, 2025

Related U.S. Application Data
(60) Provisional application No. 63/483,590, filed on Feb. 7, 2023.

(51) Int. Cl. G01N 33/49

(52) U.S. Cl. CPC G01N 33/4905

(57) ABSTRACT

Apparatus and methods for clot burst pressure measurement are disclosed herein. Specific embodiments include a pump, a pressure measurement device, and a container comprising an inlet, an outlet and an orifice. In particular embodiments, the pump is coupled to the inlet of the container, the pressure measurement device is coupled to the outlet of the container, and the orifice is configured to retain a fluid extending across the orifice.

### CROSS-REFERENCE TO RELATED APPLICATION

This application claims priority to and the benefit of U.S. Provisional Patent Application 63/483,590, filed Feb. 7, 2023, the entire contents of which is hereby incorporated by reference.

### FIELD OF THE INVENTION

Embodiments of the present disclosure relate to devices and methods for clot burst pressure measurement.

### BACKGROUND OF THE INVENTION

Hemorrhagic shock accounts for up to 40% of all trauma deaths and is responsible for a lethal triad of trauma. Due to rapid turnover of clotting factors, newly formed clots are generated in a hypocoagulable and hyperfibrinolytic state. A better understanding of clot integrity can help optimize the use of resuscitation strategies, such as permissive hypotension.

Trauma is the leading cause of death in the United States for individuals under 46. While cancer and heart disease death rates have fell, conversely trauma death rates have increased from 140,951 to 173,140 deaths from 2000 to 2010 (Rhee, 2014). Traumatic mortality is greatly influenced by poor hemorrhagic control, in both pre-hospital and hospital settings (Wade, 2006). Within trauma, hemorrhagic shock accounts for up to 40% of all deaths stemming from a lethal triad of complications.

Hemorrhagic can be induced by a blunt or penetrating injury to vasculature and at the site of injured vessel walls, exposed subendothelial collagen interacts with platelets, RBCs, and various clotting factors to generate a clot. Due to rapid turnover of clotting factors and massive volume loss associated with hemorrhage, clots in early traumatic states are generated in a hypocoagulable, hyperfibrinolytic, and hemodiluted state (Rossaint, 2010). However, as hemorrhagic shock progresses, increased production of prothrombotic factors and decreased fibrinolytic enzymatic activity induces a hypercoagulable state (Sumislawski, 2018). Consequently, clots generated in trauma experience a varying range of strength profiles compared to clots generated in hemostatic conditions. A better understanding of clot integrity in trauma can help direct acute care to reduce mortality rates and associated morbidities.

Current strategies for addressing hemorrhagic shock include permissive hypotensive resuscitation, which involves a less aggressive approach to fluid resuscitation that utilizes fewer fluids and fewer components to maintain a lower mean arterial pressure (MAP). A lower MAP is thought to prevent dislodgement of newly generated clots by reducing hydrostatic pressure (Rezende-Neto, 2010), maintaining peripheral vasoconstriction through a smaller cardiac preload (Smith, 2014), and avoiding dilutional coagulopathy in an already compromised clotting scenario (Bolliger, 2010). Previous multi-center studies have shown a reduction in overall mortality and morbidities, such as major organ failure (MOF) and acute respiratory distress syndrome (ARDS), with use of permissive hypotensive resuscitation in trauma patients (Owattanapanich, 2018). These studies are outcome-based, and do not explore the effect of individual components have on resuscitation strategy in terms of MAP.

Thromboelastography (TEG) (Haemonetics, MA) is a quantitative test that measures clotting kinetics on whole blood. Whole blood coagulates around a pin and torsional deflection is recorded, with increasing amounts of deflection indicating greater clotting kinetics. TEG-based resuscitation has been shown to positively guide resuscitation strategies to avoid coagulopathies (Suliburk, 2012), however these strategies have certain limitations. Clinical studies have shown false-negative TEG graphs for patients on heparin therapy, failing to detect heparin coagulopathy (Dunham, 2014).

### SUMMARY OF THE INVENTION

Accordingly, the embodiments of the present disclosure provide for devices and methods for measuring clot burst pressure. Particular embodiments include examples where fluid (e.g. water) is pumped over a clot and into a measurement column that is above the clot, where the fluid continuously applies increasing pressure on the clot until it bursts. It is believed that exemplary embodiments of the present disclosure can distinguish between thrombi of hypo- and hypercoagulable states by observing differences in the clot burst pressure (CBP) of conditioned groups compared to control CBP.

Specific embodiments of the apparatus comprise a sensor cartridge or container with a polymer test surface. In particular embodiments an inner surface of the container has a shallow indentation with a small hole or orifice in the center. During operation of the apparatus, a drop of fluid (e.g. blood or plasma) is placed on the indentation so that it covers the orifice, and the blood is allowed or pharmacologically induced to clot. An adhesive film may be applied to an outer surface of the container over the orifice to help hold the fluid drop in place, so the blood or plasma does not fall through the orifice before clotting. The adhesive film would be removed prior to the pressurization process. The orifice may also comprise a grid to retain the blood or plasma.

After the blood sample clots, the inner surface of the cartridge is sequentially brought to higher pressures until the clot fails and bursts through the orifice. This is signified by a sudden loss of pressure, as measured by a manometer in fluid communication with the inner volume of the container. In certain embodiments the fluid is water, and the manometer is a water column, but other embodiments may use air as the measurement fluid.

The indentation on the inner surface may be coated with one or more chemicals or proteins. These may allow enhanced or selective binding of the blood or plasma drop in a specific manner. Certain embodiments may include several such indentations and orifices which are enhanced with different chemicals, in order to provide more specific information to guide transfusion of platelets versus plasma versus clotting factors. In certain embodiments, the several indentations and orifices can be pressurized together, so that one orifice bursts first, or they can be pressurized independently, so that each orifice bursts at its own representative pressure. In particular embodiments, the several indentations and orifices might be filled individually, or they might use microfluidics to aid distribution of one blood or plasma drop to several indentations/orifices.

Exemplary embodiments of the present disclosure provide an advantage of over prior functional assays by performing the clot burst pressure test more rapidly. Once the clot reaches a desired state, the test can be run in under one minute (e.g. a few tens of seconds). Exemplary embodiments of the present disclosure also provide another advantage by physically measuring a quantity that reflects the ability of the blood clot to resist burst pressure, which is the characteristic of interest in a bleeding patient.

Exemplary embodiments of the present disclosure provide a direct, low-cost clot burst pressure (CBP) test and require a much smaller volume of blood than other tests, such as thromboelastography (TEG). Assays performed with exemplary embodiments of the present disclosure can test the impact that combinations and permutations of individual resuscitation products and clotting factors have on a patient in a clinically relevant parameter: mmHg.

Exemplary embodiments include an apparatus for measuring clot burst pressure, where the apparatus comprises: a pump; a pressure measurement device; and a container comprising an inlet, an outlet and an orifice. In certain embodiments the pump is coupled to the inlet of the container; the pressure measurement device is coupled to the outlet of the container; and the orifice is configured to retain a fluid extending across the orifice. In particular embodiments the fluid comprises blood or plasma, fibrinogen, heparin, platelets, tranexamic acid and/or thrombin. In some embodiments the fluid is coagulated. In some embodiments the orifice has a diameter greater than or equal to 0.05 inches and less than or equal to 0.20 inches. In specific embodiments the orifice is a first orifice of a plurality of orifices in the container, and each orifice in the plurality of orifices is configured to retain the fluid. In certain embodiments each orifice in the plurality of orifices has a diameter greater than or equal to 0.05 inches and less than or equal to 0.20 inches.

In particular embodiments the pump comprises a syringe. In some embodiments the pressure measurement device comprises a column comprising a measurement fluid. In specific embodiments the measurement fluid is water, saline or air. In certain embodiments the pressure measurement device comprises an aneroid manometer. In particular embodiments the pump is configured to pump the measurement fluid from the pump to the container and into the column. In some embodiments the pump is configured to pump the measurement fluid at a rate greater than or equal to 0.2 milliliters per second (mL/s) and less than or equal to 2.5 mL/s.

Specific embodiments further comprise an indicator of a level of the measurement fluid in the column. In certain embodiments the indicator is configured to detect a maximum level of the measurement fluid in the column. Particular embodiments further comprise a first conduit and a second conduit, where the first conduit is coupled to the pump and the inlet to the container, and where the second conduit is coupled to the outlet of the contain and the pressure measurement device.

In some embodiments a surface around the orifice is chemically actualized. In specific embodiments a surface around the orifice is coated with collagen, fibronectin, thrombin, fibrinogen, freeze-dried platelets and/or plasma proteins. In certain embodiments a surface around the orifice is coated with specific factors from a clotting cascade to detect deficiencies in those factors. In particular embodiments the surface around the orifice includes an indentation.

Exemplary embodiments include a method of measuring clot burst pressure, where the method comprises: applying a fluid across an orifice of a container; forming a clot from the fluid, wherein the clot extends across the orifice of the container; increasing a pressure inside the container; and measuring the pressure inside the container when the clot is dislodged from the orifice. In certain embodiments, forming the clot comprises: providing a support surface across the orifice prior to applying the fluid across the orifice; applying the fluid to the support surface; and removing the support surface after a period of time. In particular embodiments forming the clot further comprises providing a temperature inside the container greater than 25 degrees Celsius prior to removing the support surface. In some embodiments forming the clot further comprises providing a temperature inside the container greater or equal to 35 degrees Celsius and less than or equal to 40 degrees Celsius prior to removing the support surface. In specific embodiments forming the clot further comprises providing a temperature inside the container of approximately 37 degrees Celsius prior to removing the support surface. In certain embodiments the period of time is greater than or equal to 5 minutes and less than or equal to 50 minutes. In particular embodiments the time period is approximately 30 minutes.

In certain embodiments forming the clot further comprises adding heparin to the fluid. In particular embodiments forming the clot further comprises adding fibrinogen to the fluid. In some embodiments increasing the pressure inside the container comprises pumping a measurement fluid into the container. In specific embodiments measuring the pressure inside the container when the clot is dislodged from the orifice comprises measuring a column of the measurement fluid.

Any embodiment discussed with respect to one aspect of the invention applies to other aspects of the invention as well.

The embodiments in the Example section are understood to be embodiments of the invention that are applicable to all aspects of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the terms “about” and “approximately” are used to indicate that a value includes the standard deviation of error for the device or method being employed to determine the value. If the standard deviation of error for the device or method being employed to determine the value is not known, the terms are interpreted to mean plus or minus five percent of the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

These and other objects, features, and advantages of the invention will become apparent from the detailed description below and the accompanying drawings. The patent or application file contains at least one drawing executed in color. Copies of this patent or patent application publication with color drawing(s) will be provided by the Office upon request and payment of the necessary fee.

FIG. 1 illustrates a photographic overview of an exemplary embodiment according to the present disclosure.

FIG. 2 is an exploded schematic view of a container of the embodiment of FIG. 1.

FIG. 3 is section view of the container of FIG. 2.

FIG. 4 is a graph of normalized burst pressure for different examples of conditioned donor blood.

FIG. 5 illustrates photographic assembly and use of an embodiment according to the present disclosure in panels A-D.

FIG. 6 illustrates photographic assembly and use of an embodiment according to the present disclosure in panels A-L

FIGS. 7-10 illustrate graphs of burst pressure versus contraction for exemplary embodiments of the present disclosure.

### DETAILED DESCRIPTION OF THE INVENTION

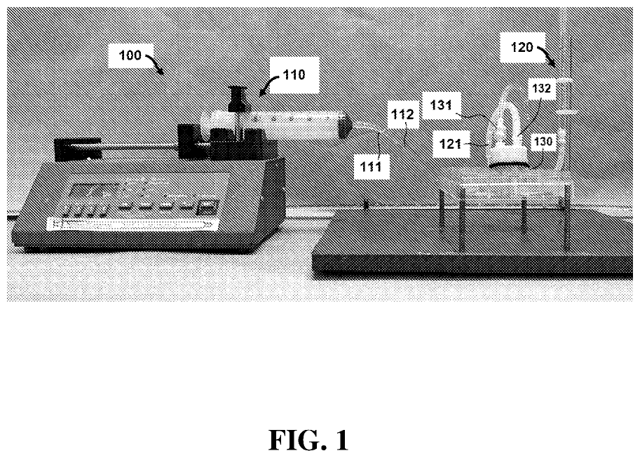

Referring now to FIGS. 1-3, an exemplary embodiment of the present disclosure comprises an apparatus 100 for measuring clot burst pressure. For purposes of clarity, not all elements are labeled with reference numbers in each figure. In the embodiment shown, apparatus 100 comprises a pump 110, a pressure measurement device 120; and a container 130 comprising an inlet 131, an outlet 132 and an orifice 135. As shown in the illustrated embodiment, pump 110 is coupled to inlet 131 of container 130 via conduit 111, while pressure measurement device 120 is coupled to outlet 132 of container 130 via conduit 121.

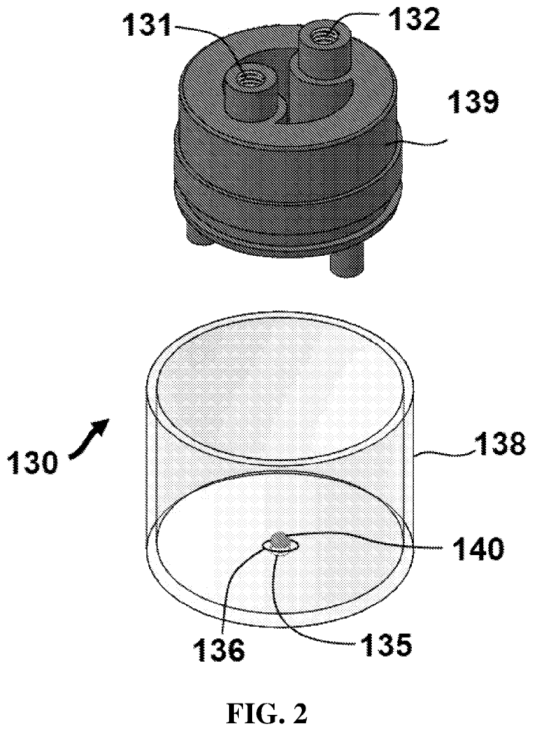

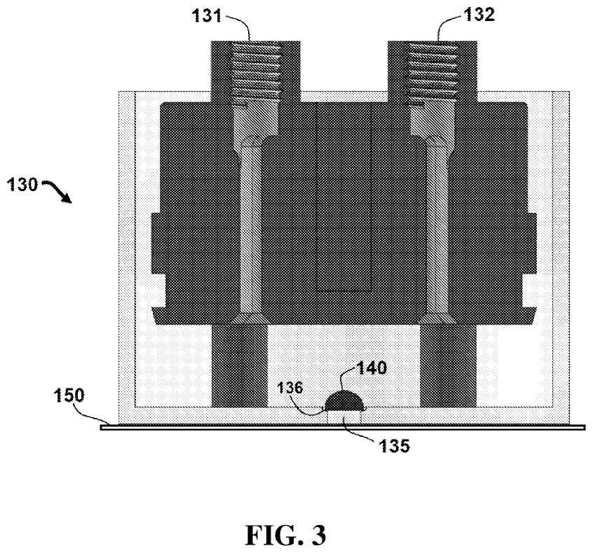

As shown in FIGS. 2 and 3, orifice 135 is configured to retain a fluid 140 extending across orifice 135. As explained further below, in certain embodiments fluid 140 may initially be in a liquid state and then coagulated to form a clot. In particular embodiments, fluid 140 may comprise blood or plasma, and may also comprise an agent to promote or impair clotting, including for example, fibrinogen or thrombin or heparin. In particular embodiments, an indentation 136 (shown in FIGS. 2 and 3) extends around orifice 135 to assist in the retention of fluid 140. In certain embodiments, indentation 136 may be chemically actualized to aid analysis of the blood sample. In particular embodiments, indentation 136 can be in a small depression that serves to hold the blood clot in place over orifice 135. The surface around orifice 135 (including the indentation, if an indentation is present) can be coated with collagen or fibronectin to aid clot adhesion and to mimic the in vivo surface. In certain embodiments the surface around orifice 135 can be coated with thrombin to generate a faster clot and therefor a quicker result. In particular embodiments the surface around orifice 135 can be coated with fibrinogen for comparison to a control clot, to detect a low fibrinogen state. Similarly, the surface around orifice 135 might be coated with freeze-dried platelets or plasma proteins, to detect deficiencies in platelets or plasma in the test sample. In certain embodiments the surface around orifice 135 can be coated with specific factors from the clotting cascade to detect deficiencies in those factors.

Container 130 comprises a body portion 138 and a cap 139. In certain embodiments, body portion 138 may be transparent such that a user can see fluid 140 during testing. In particular embodiments, a support surface 150 may be initially provided across orifice 135 to support fluid 140 during clot formation. After a period of time sufficient for fluid 140 to form a clot extending across orifice 135, support surface 150 may be removed to allow for pressure testing to determine the clot burst pressure. In particular embodiments, the period of time for fluid 140 to form a clot may be greater than or equal to 5 minutes and less than or equal to 50 minutes. In specific embodiments the period of time for fluid 140 to form a clot may be approximately 30 minutes.

In specific embodiments, the temperature inside container 130 can also be increased above ambient temperature (e.g. 25 degrees Celsius) to assist in clot formation of fluid 140. For example, the temperature inside container 130 may be increased to a temperature greater or equal to 35 degrees Celsius and less than or equal to 40 degrees Celsius prior to removing support surface 150. In a specific embodiment, the temperature inside container 130 may be increased to approximately 37 degrees Celsius prior to removing support surface 150.

In certain embodiments, after fluid 140 has been applied across orifice 135 and formed a clot (and support surface 150 removed), the pressure inside container 130 can be increased until the clot is dislodged from orifice 135. The pressure at which the clot formed from fluid 140 is dislodged from orifice 135 can be measured and documented as the clot burst pressure. In the embodiment shown, pump 140 can be operated to pump a measurement fluid 112 into container 130 via conduit 111 and inlet 131. In specific embodiments, measurement fluid 112 may be water or other suitable fluid. As pump 110 continues to pump measurement fluid 112 into container 130, measurement fluid 112 fills container 130 and flows from outlet 132 into pressure measurement device 120 via conduit 121. In the embodiment shown, pressure measurement device 120 is a column with graduated markings indicating the fluid level in the column.

As the level of measurement fluid 112 in pressure measurement device 120 is increased, the pressure exerted on the clot formed from fluid 140 is increased until the clot is eventually dislodged from orifice 135.

While the embodiment shown in FIGS. 1-3 and described above comprises a syringe pump and measurement column utilizing water as a measurement fluid, other embodiments may comprise different components. For example, other embodiments may utilize air as the measurement fluid and may comprise an air pump (e.g. a compressor) to pressurize the container containing the orifice with the clot formed from the blood or plasma drop. In addition, the pressure measurement device can be a pressure gauge or other suitable device for measuring pressure. In particular embodiments pressure measurement device 120 may be an aneroid manometer, including for example, embodiments utilizing air as measurement fluid 112.

### TEST RESULTS AND DESCRIPTION OF ONE SPECIFIC EMBODIMENT

An exemplary embodiment of the present disclosure was used to obtain test data as follows. Blood from donors (n=7) was collected into 3.2% sodium citrate tubes. Blood was divided and treated to achieve the following conditions for a hypocoagulable state: 0.10, 0.15, and 0.20 USP heparin/mL blood; for a hypercoagulable state, blood was treated with a 1.0 or 2.0 g/L addition of fibrinogen. 2 mm holes were drilled into 6-well polystyrene plates and packing tape was used to cover the bottom. Blood was recalcified with CaCl₂ and pipetted into the holes. The plates were incubated at 37 C for 30 minutes to allow for thrombus formation. Tape was removed after incubation. A pressure cap was designed with an inlet for fluid from a syringe pump and an outlet for outflow and was secured in the wells with a watertight seal. Outflow led to a vertical graduated measurement column where pressure was converted from cmH2O to mmHg for data analysis.

CBP was normalized to individual donor baseline values to account for inherent differences in clot strength. Compared to baseline values of CBP, 1.0 g/L and 2.0 g/L fibrinogen were 1.78±0.11 (p<0.0005) and 2.66±0.36 (p<0.005) times as strong, respectively. Compared to baseline values, 0.10, 0.15, 0.20 USP heparin/mL were 0.54±0.19 (p<0.005), 0.41±0.17 (p<0.0005), 0.25±0.02 (p<0.0005) times as strong, respectively. Data was analyzed using one-way ANOVA for multiple comparisons with Tukey.

The investigators were able to create a model that allows for testing of clot strength in a parameter, burst pressure, not seen in existing devices such as TEG. The CBP model was able to show significant differences between control, hypercoagulable, and hypocoagulable groups across multiple donors. Future exploration of the device can involve the use of blood from trauma patients to better understand trauma pathophysiology.

The device comprises four primary components as shown in FIG. 1: a syringe pump, a pressure cap, a 6-well plate assembly, and a water column. A Programmable Syringe Pump was programmed to pump a 100 cc syringe at 1 mL/s at a continuous rate. The syringe pump is coupled to a pressure cap assembly.

The pressure cap assembly shown in FIGS. 2 and 3 directs fluid flow directly over a clot and prevents pressure loss to the environment. The cap has two female ports allowing for an inlet connection from the syringe pump and an outlet connecting to the water column. The cap has three legs to avoid direct contact with a clot. An O-Ring (Dash 242) fits in the groove found around the pressure cap ensuring a snug fit in a standard 6-well plate. As fluid enters the pressure cap, it is directed into a compartment where a formed clot is suspended. Backflow fluid rises up a 150 cm water column created from clear PVC pipe marked at 5 cm intervals. All connections used Luer locks and barbed connections to prevent fluid loss.

A 20″×20″ HDPE board was used as the base of the device, which contained the 6-well plate assembly and water column. 4, 2″ screws were used to elevate the 6-well plate assembly to allow for easy visual inspection after trial runs. A single 5/64″ hole was drilled into the centers of each well in a 6-well plate. 220-grit sandpaper was used in a circular manner on the inner surface of the well-plate to provide a sheared surface for platelet adhesion. Clear packing tape was used to cover the holes on the bottom of the plate prior to the start of the assay.

### Blood Sample Collection

Venipuncture whole blood (WB) was collected from donors (male and female) who were at least 18 years of age and had not taken anticoagulant medication in the past 14 days. Blood was drawn from the antecubital fossa using a 19G needle into a standard blue top 3.2% sodium citrate vacutainer tube. Tubes were placed on a rocker at room temperature prior to use in the assay, but all assay runs were initiated within 30 minutes of blood draw.

### Blood Conditioning

WB was conditioned to mimic hyper and hypocoagulable states. RiaSTAP a lyophilized fibrinogen concentrate, was used to generate a hypercoagulable condition. WB was conditioned with a 1.0 mg/mL or 2.0 mg/mL bolus of fibrinogen. Heparin sodium salt (Sigma-Aldrich, OH) was reconstituted and added to WB at 0.10, 0.15, 0.20 USP/mL amounts to generate a hypocoagulable state. 6 mM of CaCl₂) was added to each conditioned blood group and a control group to recalcify and promote coagulation.

### CBP Assay

35 μL of conditioned or control WB was placed in the 5/64″ hole above the packing tape in each well of the 6-well plates. The entire plate was incubated at 37 C for 30 min with the plate lid on. After 30 min, the plate was removed from the incubator and the packing tape on the bottom was removed. The pressure cap was placed in a single well in a spiral manner and an air-tight seal was created with the O-Ring. One port of the pressure cap was connected to the syringe pump and the other port to the vertical water column. The syringe pump was turned on and the assay was run until the clot was dislodged or burst. The maximum height of the water column was recorded as the clot burst pressure in cm. This process was repeated for each trial, with the syringe refilled with DIH2O in between runs. Data in cmH2O was converted to mmHg.

### Efficacy Testing

Testing to verify the efficacy of the assay was performed. Blood used in verification trials came from 3 donors and trials were run for 6 replicates. Initial trials sought to identify the ideal syringe pump flow rate, an optimal diameter in the well plate for thrombus formation, incubation time for thrombus formation, the use of whole blood vs. conditioned plasma, and materials to condition donor blood. Tables 1A-1D below detail the combinations tested and a summary of remarks.

TABLE 1A

| | | |
|---|---|---|
| Syringe Pump Flow | Time to completion | Difference Between |
| Rate | (single trial) | All Groups in CBP |

| | | | | |
|---|---|---|---|---|
| 0.25 | mL/s | 3 | minutes | No |
| 0.5 | mL/s | 1.5 | minutes | No |
| 1 | mL/s | 0.75 | minutes | No |
| 1.5 | mL/s | 0.43 | minutes | No |

| | | | |
|---|---|---|---|
| 2 | mL/s | Premature clot | Yes |
| | | dislodgment | |

TABLE 1B

| Diameter of Hole for | | Difference Between |
|---|---|---|
| Clot Formation | Clot Integrity | Groups in CBP |
| 5/64″ | Stable clot, minimal | No |
| | blood volume | |
| 8/64″ | Stable clot, large | No |
| | volume required | |
| 10/64″ | Stable clot, large | No |
| | volume required | |

TABLE 1C

| Incubation Time | | Difference Between |
|---|---|---|
| ( 5/64″) Clot | Clot Integrity | All Groups in CBP |
| 15 minutes | Clot dislodged | Yes |
| | during tape removal | |
| 20 minutes | Clot not fully formed | No |
| 30 minutes | Stable clot | No |
| 40 minutes | Stable clot | No |
| 45 minutes | Clot had dried out | Yes |

TABLE 1D

| Whole | | | | |
|---|---|---|---|---|
| Blood w/ | Whole Blood | ATIII-deficient | | Difference Between |
| RiaSTAP | w/Heparin | plasma w/ATIII | Clot Integrity | All Groups in CBP |
| 0.5 g/L | — | — | Stable Clot | Yes |
| 1.0 g/L | — | — | Stable Clot | Yes |
| 1.5 g/L | — | — | Stable Clot | Yes |
| 2.0 g/L | — | — | Stable Clot | Yes |
| 3.0 g/L | — | — | Stable Clot | No |
| — | 0.35 USP/mL | — | No Clot Formation | N/A |
| — | 0.25 USP/mL | — | Porous Clot | N/A |
| — | 0.20 USP/mL | — | Weakened Clot | Yes |
| — | 0.15 USP/mL | — | Weakened Clot | Yes |
| — | 0.10 USP/mL | — | Weakened Clot | Yes |
| — | — | 80 mg/L | Porous Clot | N/A |
| — | — | 120 mg/L | Porous Clot | N/A |
| — | — | 160 mg/L | Porous Clot | N/A |

### CBP Data

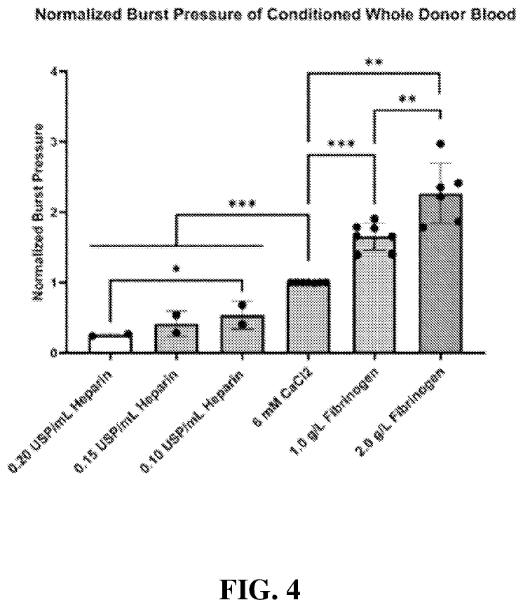

CBP was normalized to control CBP measurements to account for inherent differences in baseline clotting factors for each donor shown in FIG. 4. FIG. 4 shows increasing concentrations of heparinized blood creates weaker clots compared to control (***p<0.0005) and within groups (*p<0.05). Increasing concentrations of spiked fibrinogen generates stronger clots compared to control at 1.0 g/L (***p<0.0005), 2.0 g/L (**p<0.005), and within groups (**p<0.005). Seven donors were used for the control, 1.0 g/L, 2.0 g/L fibrinogen groups, and 2 of those 7 were used for the heparinized conditions. All trials were performed in triplicate. Compared to baseline values of CBP, mean WB conditioned with 1.0 g/L or 2.0 g/L fibrinogen boluses were 1.78±0.11 (p<0.0005) and 2.66±0.36 (p<0.005) times as strong, respectively. Compared to baseline values of CBP, mean WB conditioned with 0.10, 0.15, or 0.20 USP heparin/mL were 0.54±0.19 (p<0.005), 0.41±0.17 (p<0.0005), 0.25±0.02 (p<0.0005) times as strong, respectively. All conditions were significantly different compared to baseline, and fibrinogen boluses displayed a dose-dependent strength increase within the hypercoagulable condition. The heparin boluses displayed a dose-dependent weakness between the 0.10 and 0.20 USP heparin/mL group and an overall decreasing trend throughout the group.

The aim of this project was to create a low-cost prototype that could rapidly measure clot strength in a clinically relevant manner. Development of the device occurred in stages, with iterations improving certain facets of the design. Additionally, optimization of the assay also provided certain challenges. 5 key areas were addressed: syringe pump flow rate, type of fluid, hole diameter, clot incubation time, and blood conditioning to generate a dose-dependency curve (Tables 1A-1D).

Samples run in TEG average around 45 minutes to run to completion, thus a similar or smaller time constraint was sought for this device. A flow rate was chosen that would allow for timely testing, while not being too powerful as to cause shearing of the clot. The device measurements should only be influenced by compressive forces normal to the clot for most accurate pressure readings. A flow rate of 1 mL/s was ultimately chosen as this met both criteria (Note: 1.5 mL/s was also a viable flow rate).

Based on previous literature about the use of microvolumes of blood for clots kinetic testing, 35 μL of blood was used as an initial starting point (George, 2018). To generate clots for the 8/64″ and 10/64″, the square: cube relationship was observed, and volumes of 89.6 μL and 140 μL of blood were used to generate clots. No significant difference in CBP was observed between diameters and to preserve blood volume from donors, a diameter of 5/64″ was used in all following tests. Furthermore, no difference in CBP was observed when using 1.0×PBS (pH=7.4) vs DIH2O as the fluid in the syringe pump, and to reduce costs, DIH2O was used in all tests.

Specific embodiments of the present disclosure device rely on the formation of a non-porous clot. While the clot is not continuously perfused during incubation, it is important to establish an ideal incubation time that generates a clot that is neither immature in strength, nor evaporated due to excess time in the incubator. For the 5/64″ diameter clots, 30 minutes of incubation at 37 C achieved these goals. In trials with longer times of incubation, clots were too brittle and had dried out. During testing, these clots would fracture along edges or simply fall out of the wells with minimal pressure. Conversely, in shorter time points, clots had not achieved sufficient time to complete fibrinogen to fibrin crosslinking, and fluid would permeate through the clot at minimal pressure. These immature clots were not solid and displayed a more viscous consistency. A successful clot operated between these extremes.

In physiological conditions, clot shape can be influenced by multiple factors including fluid velocity, tissue factor exposure, venous shearing, and relative availabilities of clotting factors in the microenvironment (Govindarajan, 2018). However, to compare strengths of clots between donors and within conditions, clots needed to be uniformly shaped. Careful placement of blood pre-incubation ensured that clots would burst at a midpoint, indicating that pressure was evenly distributed normal to the surface of the clot, and that misshapen clot formation did not influence premature dislodgment.

Fibrinogen consists of 3 pairs of polypeptide chains. Activated thrombin from the coagulation cascade cleaves fibrinopeptides from 2 of the polypeptide chains allowing soluble fibrinogen to crosslink and form staggered fibrin polymers. Factor XIIIa (also activated by thrombin) then cross-links glutamine residues on these staggered fibrin polymers with one another generating a multi-layered clot. Elevated amounts of fibrinogen increase clot strength, and studies have shown the use of fibrinogen concentrate in trauma to restore hemostasis and increase clot strength (Machlus, 2011, Schochl, 2009). Consequently, to test efficacy of the CBP device at hypercoagulable or increased clotting states, boluses of fibrinogen were added to baseline blood samples. Normal physiological levels of fibrinogen range from 2.0 g/L to 4.5 g/L (Levy, 2015). The timeframe of this experiment did not permit testing the donors for baseline fibrinogen levels. All values were normalized to individual baseline CBP to account for inherent different in clotting factors. The spiked bolus values of 1.0 g/L and 2.0 g/L represent an increase of 50% and 100%, respectively, on the lowest end of the physiological spectrum. As seen in FIG. 4, fibrinogen linearly increased the strength of clots in a dose-dependent manner.

Conversely, heparin was used to generate weaker clots representing a hypocoagulable state. Heparin binds to antithrombin III (ATIII) and causes a conformational change increasing enzyme activity. Activated ATIII inactivates thrombin and Factor Xa causing a direct halt in clot formation and subsequent coagulation activity. A 5000 USP/mL dose of heparin sodium for a standard patient (5 L volume) is equivalent to a 0.35 USP/mL dose for the 35 μL of blood used in this device. However, this dose completely inhibits clot production. The dose was reduced to a 0.20-0.10 USP/mL range, which allowed for a weaker, but stable clot to form. Heparin was chosen to weaken clots because its mechanism of action is well understood, it is indicated for use for many coagulopathies, and because of previous literature detailing limitations of TEG in detecting heparin-related coagulopathies (Dunham, 2014). The device was able to record significant readings at a hypocoagulable state, indicating its efficacy when using samples conditioned with heparin.

ATIII deficient plasma with spiked ATIII boluses was considered to generate a dose dependent curve. However, plasma formed clots were porous and would not resist fluid pressure properly in the device. Recent studies have shown that red blood cells (RBCs) play in important role in clot formation and hemostasis (Gillespie, 2021). RBCs serve as the plug that the cross-linked fibrin mesh traps against the exposed vasculature, generating a non-porous clot (Weisel, 2019). The use of the plasma without RBCs in this device further validates this concept.

### Test Results and Description of Second Specific Embodiment

### Assembly:

Transwell inserts without the pre-loaded membrane were used as a support structure (Corning™ Transwell™ Multiple Well Plate with Permeable Polycarbonate Membrane Inserts, 6/plate, Cat #07-200-165 Corning™ 3412). The membrane was removed after (other) use and the insert was reused.

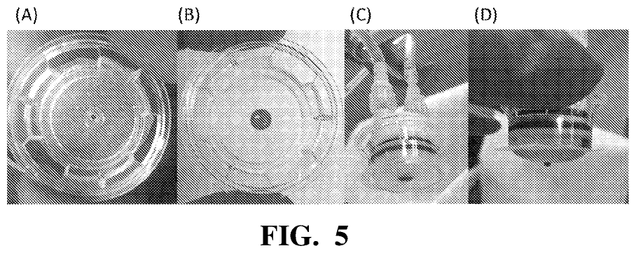

An elastic collagen membrane was attached to the bottom of the transwell and secured to hold in place with glue (Bondic—UV LED Welding Starter Kit with Liquid Plastic). A 1.5-mm hole was made in the center of the membrane and a piece of acrylic with 1-mm hole in the center was glued to the collagen membrane such that the centers of the two holes coincide (FIG. 5A).

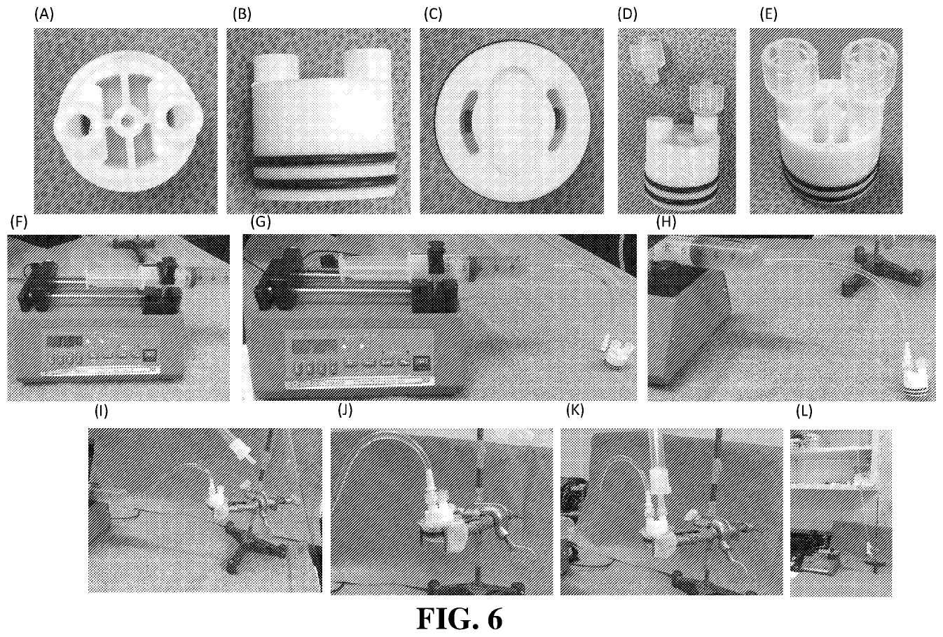

The top (FIG. 6 A,B,C) was attached through 2 Luer locks (FIGS. 6 D and F) with one end (designated as an inlet) to the syringe pump (FIG. 6F) and with another end (designated as an outlet) to a pressure column (FIG. 6 K,L). The connection between Luer lock and plastic tubing was Luer adapter to ensure that the tubing is securely attached.

The top was inserted into the transwell (FIG. 6, I, J) and secured in place with a clamp. The connector leading to the pressure column was attached to the designated outlet (FIG. 5C, FIG. 6 K). The pump was turned ON and the media from the syringe was pumped through the top into the transwell with blood clot and out into the pressure column.

Troubleshooting and limitations of materials and procedures noted in testing:
1. The collagen membrane can be stretched significantly, so the original 1 mm hole becomes much larger, resulting in loss of blood clot.
2. The attachment of acrylic insert to the collagen membrane helped keeping the opening (1 mm) always of the same size, repeatable and reproducible.
3. The collagen membrane is water-resistant. If a hole was not made in the collagen membrane, but instead an intact membrane was used to flow the media, the membrane would stretch, but not leak.
4. The transwell has to be flipped upside down immediately after the blood is pipetted. If it is not flipped, the blood will seep out before fully coagulating and sample will be lost.

### Procedure:

Blood sample (35 uL) was pre-incubated with activators or inhibitors of platelet activation for 15-30 minutes. The blood then was mixed with 6 mM calcium and immediately pipetted onto the acrylic to allow for attachment and clot stabilization. The transwell was immediately flipped upside down to “hang” the blood drop. The blood was allowed to clot for 15 minutes (FIG. 5B). The top was inserted into the transwell, after which the connecting tubes to the syringe pump and pressure column were attached.

Media at flow rate 15 ml/min was fed into the transwell through the top and the pressure of clot burst was noted at the pressure column (FIG. 6L).

### Data:

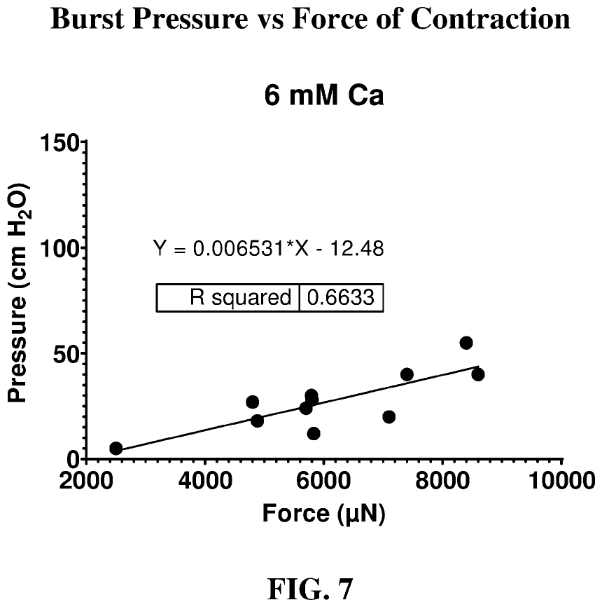

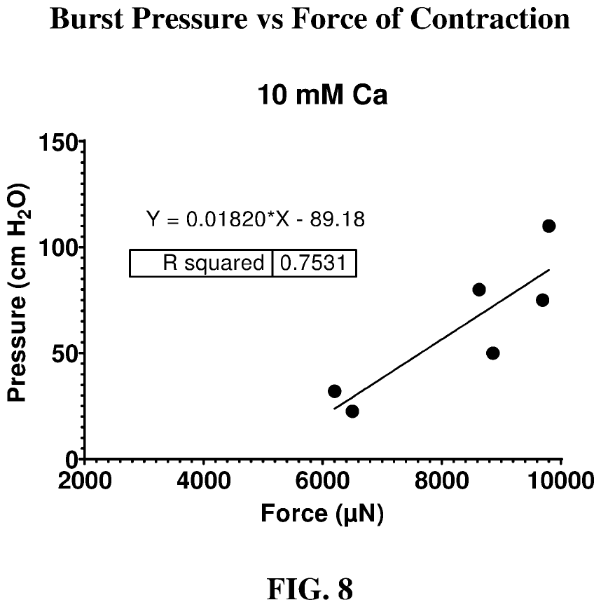

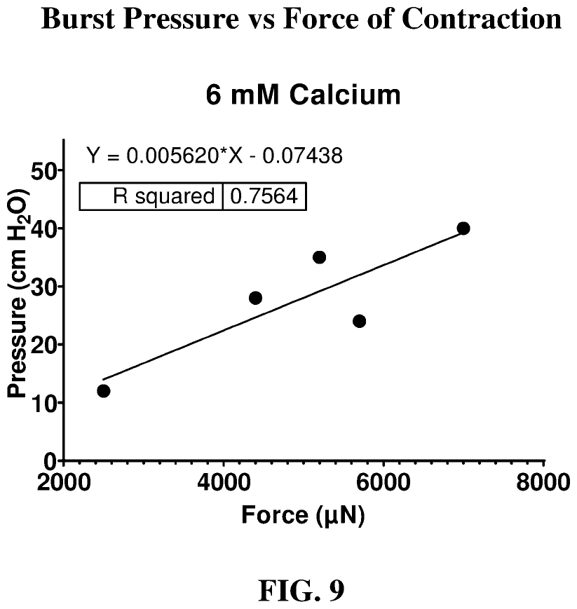

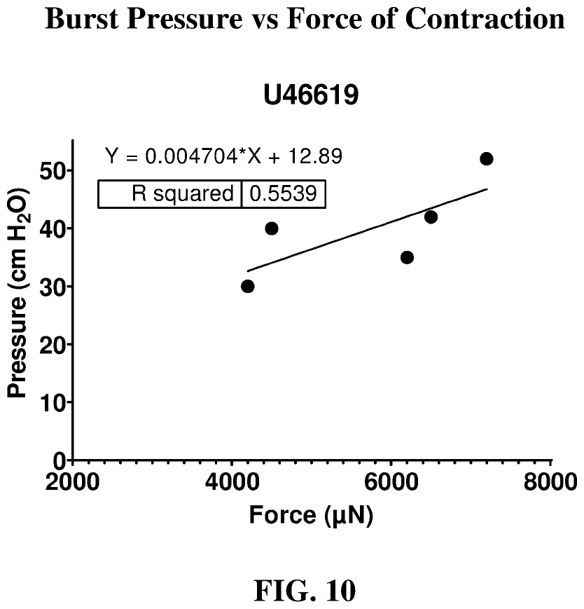

Blood from trauma samples and healthy controls was activated with 6 mM calcium (FIG. 7), 10 mM calcium (FIG. 8), or 250 pM U46619 with 6 mM calcium (FIGS. 9 and 10). U46619 causes shape change without inducing aggregation. TxA2 receptor in platelets is TPalpha only, no beta, and a lower concentration of U46619 is required to induce shape change than to cause aggregation.

Exemplary embodiments of the present disclosure may include transition to a digital data collection system linked to a microcontroller for user input/output, alteration of the water column to a peristaltic pump to mimic the pulsatile flow seen in vasculature, and addition of a channel for constant perfusion of the clot during incubation.

Exemplary embodiments of the present disclosure may also investigate the use of additional clotting factors and various resuscitation therapies on clot strength. The investigators anticipate identifying the CBP of whole blood samples from Level 1 Trauma Patients and correlating those values to TEG data and mortality and morbidities. Expansion of the data set will help verify the efficacy of the device amongst a larger cohort.

### CONCLUSION

Exemplary embodiments of the present disclosure are able to show significant differences between control, hypercoagulable, and hypocoagulable groups across multiple donors. Exemplary embodiments of the present disclosure are easy-to-use, low cost, and investigate certain parameters not seen in established devices, such as TEG. A better understanding of clot strength can help guide resuscitation strategies and improve outcomes for patients experiencing coagulopathies. Identifying the exact impact that a clotting factor/therapy or combination of factors has on the strength of a clot can prevent immature clot dislodgment during resuscitation and potentially save lives.

It should be observed that while the foregoing detailed description of various embodiments of the present invention is set forth in some detail, the invention is not limited to those details and devices, kits and methods according to the invention can differ from the disclosed embodiments in numerous ways. It will be appreciated that the functions disclosed herein as being performed by particular embodiments may be performed differently in an alternative embodiment. It should be further noted that functional distinctions are made above for purposes of explanation and clarity; structural distinctions in a system or method according to the invention may not be drawn along the same boundaries. Hence, the appropriate scope hereof is deemed to be in accordance with the claims as set forth below.

What is claimed is:

1. An apparatus for measuring clot burst pressure, the apparatus comprising:
a pump;
a pressure measurement device; and
a container comprising an inlet, an outlet and an orifice, wherein:
the pump is coupled to the inlet of the container;
the pressure measurement device is coupled to the outlet of the container; and
the orifice is configured to retain a fluid extending across the orifice.

2. The apparatus of claim 1 wherein the fluid comprises blood or plasma.

3. The apparatus of claim 1 or claim 2 wherein the fluid comprises fibrinogen.

4. The apparatus of any one of claims 1-3 wherein the fluid comprises heparin.

5. The apparatus of any one of claims 1-4 wherein the fluid comprises platelets.

6. The apparatus of any one of claims 1-5 wherein the fluid comprises tranexamic acid.

7. The apparatus of any one of claims 1-6 wherein the fluid comprises thrombin.

8. The apparatus of any one of claims 1-7 wherein the fluid is coagulated.

9. The apparatus of any one of claims 1-8 wherein the orifice has a diameter greater than or equal to 0.05 inches and less than or equal to 0.20 inches.

10. The apparatus of any one of claims 1-9 wherein:
the orifice is a first orifice of a plurality of orifices in the container; and
each orifice in the plurality of orifices is configured to retain the fluid.

11. The apparatus of any one of claims 1-10 wherein each orifice in the plurality of orifices has a diameter greater than or equal to 0.05 inches and less than or equal to 0.20 inches.

12. The apparatus of any one of claims 1-11 wherein the pump comprises a syringe.

13. The apparatus of any one of claims 1-12 wherein the pressure measurement device comprises a column comprising a measurement fluid.

14. The apparatus of claim 13 wherein the measurement fluid is water.

15. The apparatus of claim 13 wherein the measurement fluid is saline.

16. The apparatus of claim 13 wherein the measurement fluid is air.

17. The apparatus of claim 16 wherein the pressure measurement device comprises an aneroid manometer.

18. The apparatus of any one of claims 14-16 wherein the pump is configured to pump the measurement fluid from the pump to the container and into the column.

19. The apparatus of claim 18 wherein the pump is configured to pump the measurement fluid at a rate greater than or equal to 0.2 milliliters per second (mL/s) and less than or equal to 2.5 mL/s.

20. The apparatus of claim 19 further comprising an indicator of a level of the measurement fluid in the column.

21. The apparatus of claim 20 wherein the indicator is configured to detect a maximum level of the measurement fluid in the column.

22. The apparatus of any one of claims 1-21 further comprising:
a first conduit; and
a second conduit, wherein:
the first conduit is coupled to the pump and the inlet to the container; and
the second conduit is coupled to the outlet of the contain and the pressure measurement device.

23. The apparatus of any one of claims 1-22 wherein a surface around the orifice is chemically actualized.

24. The apparatus of any one of claims 1-22 wherein a surface around the orifice is coated with collagen.

25. The apparatus of any one of claims 1-22 wherein a surface around the orifice is coated with fibronectin.

26. The apparatus of any one of claims 1-22 wherein a surface around the orifice is coated with thrombin.

27. The apparatus of any one of claims 1-22 wherein a surface around the orifice is coated with fibrinogen.

28. The apparatus of any one of claims 1-22 wherein a surface around the orifice is coated with freeze-dried platelets or plasma proteins.

29. The apparatus of any one of claims 1-22 wherein a surface around the orifice is coated with specific factors from a clotting cascade to detect deficiencies in those factors.

30. The apparatus of any one of claims 23-29 wherein the surface around the orifice includes an indentation.

31. A method of measuring clot burst pressure, the method comprising:
applying a fluid across an orifice of a container;
forming a clot from the fluid, wherein the clot extends across the orifice of the container;
increasing a pressure inside the container; and
measuring the pressure inside the container when the clot is dislodged from the orifice.

32. The method of claim 31 wherein forming the clot comprises:
providing a support surface across the orifice prior to applying the fluid across the orifice;
applying the fluid to the support surface; and
removing the support surface after a period of time.

33. The method of claim 32 wherein forming the clot further comprises providing a temperature inside the container greater than 25 degrees Celsius prior to removing the support surface.

34. The method of claim 32 wherein forming the clot further comprises providing a temperature inside the container greater or equal to 35 degrees Celsius and less than or equal to 40 degrees Celsius prior to removing the support surface.

35. The method of claim 32 wherein forming the clot further comprises providing a temperature inside the container of approximately 37 degrees Celsius prior to removing the support surface.

36. The method of any one of claims 32-35 wherein the period of time is greater than or equal to 5 minutes and less than or equal to 50 minutes.

37. The method of claim 36 wherein the time period is approximately 30 minutes.

38. The method of any one of claims 31-37 wherein forming the clot further comprises adding heparin to the fluid.

39. The method of any one of claims 31-38 wherein forming the clot further comprises adding fibrinogen to the fluid.

40. The method of any one of claims 31-39 wherein increasing the pressure inside the container comprises pumping a measurement fluid into the container.

41. The method of any one of claims 31-40 wherein measuring the pressure inside the container when the clot is dislodged from the orifice comprises measuring a column of the measurement fluid.
